(19) United States
(12) Patent Application Publication
(10) Pub. No.: US 2025/0235160 A1
(43) Pub. Date: Jul. 24, 2025

(54) BODY NOISE SIGNAL PROCESSING

(71) Applicant: Cochlear Limited, Macquarie University, NSW (AU)

(72) Inventors: Thomas Leroux, Valbonne (FR); Christian Kolind, East Killara, NSW (AU)

(21) Appl. No.: 18/855,908

(22) PCT Filed: Apr. 12, 2023
(86) PCT No.: PCT/IB2023/053751

§ 371 Date: Oct. 10, 2024

Related U.S. Application Data
(60) Provisional application No. 63/332,549, filed on Apr. 19, 2022.

(51) Int. Cl. A61B 5/00; A61B 5/024; A61B 7/02; A61B 7/04; A61N 1/36

(52) U.S. Cl. CPC A61B 5/686; A61B 5/02444; A61B 5/7203; A61B 5/7221; A61B 7/023; A61B 7/04; A61B 2562/0219; A61N 1/36038

(57) ABSTRACT

Presented herein are techniques for detecting the heartbeat of the recipient of an implantable medical device. The implantable medical device includes sensors for capturing audio signals and vibrations signals from the body of the recipient. A heartbeat detection module filters at least the vibration signals to generate a heartbeat signal representing the heartbeat of the recipient.

### BACKGROUND

### Field of the Invention

The present invention relates generally to body noise signal processing.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, an implantable medical device is provided. The implantable medical device comprises: an implantable microphone arrangement configured to be implanted in a recipient, wherein the implantable microphone arrangement comprises an acoustic sensor configured to detect acoustic signals and a vibration sensor configured to detect vibration signals; and a heartbeat detection module configured to filter at least the vibration signals to generate a heartbeat signal representing a heartbeat of the recipient.

In another aspect, a method is provided. The method comprises: detecting signals at first and second sensors of an implantable microphone arrangement configured to be implanted in a recipient, wherein the signals detected at one or more of the first and second sensors include body noises of the recipient and acoustic sound signals; determining, based at least on the body noises of the recipient, a heartbeat of the recipient; and generating an output representing the heartbeat of the recipient.

In another aspect, a method is provided. The method comprises: obtaining data from at least one sensor, the data comprising an acoustic signal of external sounds and a body noise signal of internal vibrations; determining a body environment based on the acoustic signal and the body noise signal; generating a validation signal indicating whether the body environment permits generation of a valid heartbeat signal; and based on a determination that the validation signal indicates the body environment permits the generation of the valid heartbeat signal, filtering the body noise signal to generate the valid heartbeat signal.

In another aspect, an acoustic sensor configured to measure an acoustic signal in a recipient; a vibration sensor configured to measure a body noise signal of the recipient; and a heartbeat detection module configured to: determine an activity classification of the recipient based on the acoustic signal and the body noise signal; and based on a determination that the activity classification enables a valid heartbeat signal for the recipient, filter the body noise signal to generate the valid heartbeat signal.

In another aspect, one or more non-transitory computer readable storage media encoded with software comprising computer executable instructions are provided. The software is executed operable to cause a processor of an implantable medical device to perform operations including: obtaining data from at least one sensor of the implantable medical device, the data comprising an acoustic signal of external sounds and a body noise signal of internal vibrations; determining a body environment based on the acoustic signal and the body noise signal; and based on a determination that the body environment permits generation of a valid heartbeat signal, filtering the body noise signal to generate the valid heartbeat signal.

In another aspect a device is provided. The device comprises: a microphone configured to measure an acoustic signal in a recipient; an accelerometer configured to measure a body noise signal of the recipient; one or more processors configured to determine, based at least on the a body noise signal of the recipient, a heartbeat of the recipient; and a communications module configured to provide the valid heartbeat signal to a second device.

### BRIEF DESCRIPTION OF THE DRAWINGS

Embodiments of the present invention are described herein in conjunction with the accompanying drawings, in which:

FIG. 1A is a schematic diagram illustrating a cochlear implant, in accordance with certain embodiments presented herein;

FIG. 1B is a block diagram of the cochlear implant of FIG. 1A;

FIG. 2 is a block diagram illustrating operations of a signal processing system, in accordance with certain embodiments presented herein;

FIG. 3 is a block diagram illustrating operations of heartbeat signal validation system, in accordance with certain embodiments presented herein;

FIG. 4A is a block diagram illustrating operations of binaural audio signal processing system, in accordance with certain embodiments presented herein;

FIG. 4B is a block diagram illustrating operations of bimodal audio signal processing system, in accordance with certain embodiments presented herein;

FIG. 4C is a block diagram illustrating operations of an audio signal processing system, in accordance with certain embodiments presented herein;

FIG. 5 is a graph illustrating an example heartbeat signal filtered from a body noise signal, in accordance with certain embodiments presented herein;

FIG. 6 is a flowchart of an example method, in accordance with certain embodiments presented herein;

FIG. 7 is another flowchart of an example method, in accordance with certain embodiments presented herein;

FIG. 8 is another flowchart of an example method, in accordance with certain embodiments presented herein;

FIG. 9 is a schematic diagram illustrating a stimulation system with which aspects of the techniques presented herein can be implemented; and

FIG. 10 is a schematic diagram illustrating a vestibular stimulator system with which aspects of the techniques presented herein can be implemented; and

FIG. 11 is a schematic diagram illustrating a retinal prosthesis system with which aspects of the techniques presented herein can be implemented.

### DETAILED DESCRIPTION

Presented herein are techniques configured to measure a heart rate and intensity in a recipient, via at least two sensors, such as two implantable sensors. In certain embodiments, the two sensors are located in the head of a recipient (e.g., far from the heart) and include a vibration sensor (e.g., an accelerometer) that is sensitive enough to measure vibration resulting from the heartbeat of the recipient. For example, certain implantable hearing devices typically use an accelerometer to remove body noise from an acoustic microphone input in order to enhance the input signal for the implantable hearing device. The techniques presented herein analyze the accelerometer signal to determine/measure heartbeat signal (e.g., measure the heart rate and intensity) which, in turn, can be used to evaluate/monitor cardiac function of the recipient.

In one example, the measurement of a heartbeat signal may occur separately from processing related to the main function of an implantable medical device. For instance, an implantable hearing device may measure the heartbeat signal without stimulation of the hearing in conjunction with ipsilateral or contralateral hearing aids for measuring stress or listening effort. Additionally, an implantable hearing device may send heartbeat information to a remote device (e.g., fitting system) even though the implantable hearing device is not currently stimulating.

In certain examples, the measured of the heartbeat signal may be used at fitting to measure stress due to loud stimulation or listening effort. The variation of heart rate and/or intensity of the heartbeats may be measured with the knowledge that the person is typically standing still or at rest during a fitting session. The heartbeat signal may be used as input to a classifier used to refine environment detection. Additionally, a bimodal hearing device may use the heartbeat signal for classification of the environment.

Several filtering methods may be applied to the signal from the vibration sensor in order to extract the heartbeat signal. For instance, low pass filtering, bandpass filtering, Fast Fourier Transform (FFT) analysis, Kalman filtering, and/or adaptive filtering. Extracting the heart rate and intensity may also use techniques such as a smoothing average, counters, mean, and/or variance. Additionally, the implantable medical device may use one or more options to validate or invalidate a heartbeat signal, such as broadband/narrowband amplitude of the vibration sensor signal (e.g., measure the heartbeat only when the vibration amplitude is low, or only for low frequencies), using preprocessing variables (e.g., body noise level, adaptive filter, etc.), or combining the microphone signal with the vibration sensor signal.

In certain examples, if the vibration amplitude exceeds a predetermined threshold, then the implanted device may determine that measuring a valid heartbeat signal is not possible. In other examples, the implantable medical device may determine, for example, whether the heartbeat signal valid or corrupted based on the body environment (e.g., a vibration sensor measures all body noises and, as such, some body environments may not be suitable for use in measuring a heartbeat signal). The validation of the heartbeat signal based on the body environment may be broadband or narrowband for a finer detection of the validity of the heartbeat signal.

Merely for ease of description, the techniques presented herein are primarily described with reference to a specific implantable medical device system, namely a cochlear implant system. However, it is to be appreciated that the techniques presented herein can also be partially or fully implemented by other types of implantable medical devices. For example, the techniques presented herein can be implemented by other hearing devices or auditory prosthesis systems that include, e.g., one or more other types of auditory prostheses, such as middle ear auditory prostheses, bone conduction devices, direct acoustic stimulators, electro-acoustic prostheses, auditory brain stimulators, combinations or variations thereof, etc. The techniques presented herein can also be implemented by conventional hearing aids or dedicated tinnitus therapy devices and tinnitus therapy device systems. As used herein, the term “hearing device” is to be broadly construed as any device that delivers sound signals to a user in any form, including in the form of acoustical stimulation, mechanical stimulation, electrical stimulation, etc. As such, a hearing device can be a device for use by a hearing-impaired person (e.g., hearing aid, auditory prosthesis, tinnitus therapy devices, etc.) or a device for use by a person with normal hearing (e.g., consumer devices that provide audio streaming, consumer headphones, earphones and other listening devices).

In certain circumstances, the techniques presented herein can also be implemented by balance prostheses (e.g., vestibular implants), retinal or other visual prostheses, cardiac devices (e.g., implantable pacemakers, defibrillators, etc.), seizure devices, sleep apnea devices, electroporation devices, spinal cord stimulators, deep brain stimulators, motor cortex stimulators, sacral nerve stimulators, pudendal nerve stimulators, vagus/vagal nerve stimulators, trigeminal nerve stimulators, diaphragm (phrenic) pacers, pain relief stimulators, other neural, neuromuscular, or functional stimulators, etc. The techniques presented herein can also be implemented with throat microphones and/or acoustic or vibration sensors located elsewhere in the body (e.g., not in the head of the recipient), as well as non-implantable sensors, such as mouth microphones, ear canal microphones, etc.

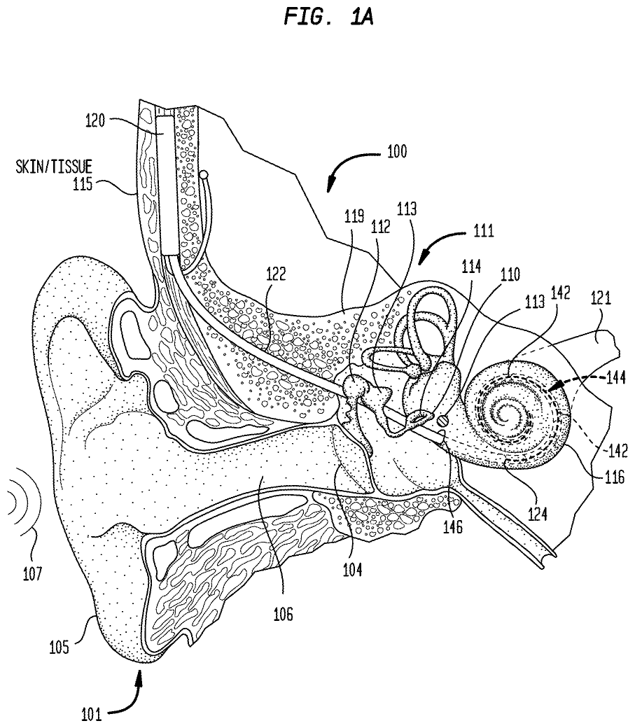

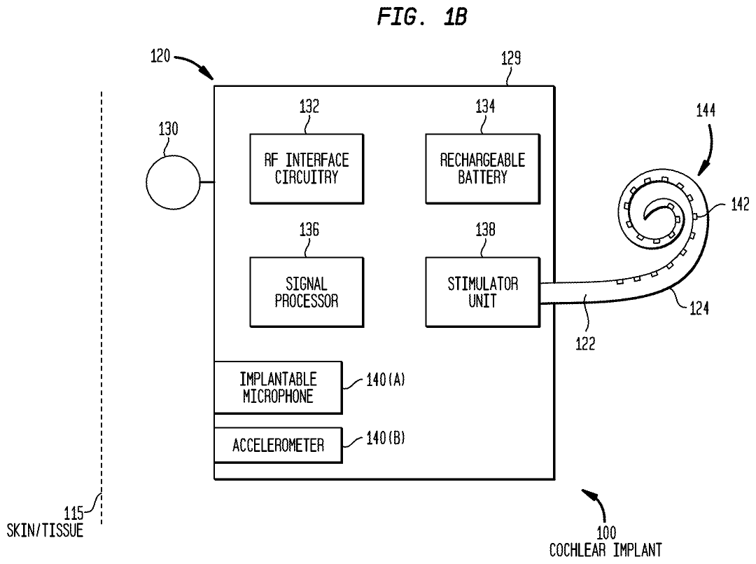

FIG. 1A is schematic diagram of an exemplary cochlear implant 100 configured to implement embodiments of the present invention, while FIG. 1B is a block diagram of the cochlear implant 100. For ease of description, FIGS. 1A and 1B will be described together.

Shown in FIG. 1A is an outer ear 101, a middle ear 102 and an inner ear 103 of the recipient. In a fully functional human hearing anatomy, the outer ear 101 comprises an auricle 105 and an ear canal 106. Sound signals 107, sometimes referred to herein as acoustic sounds or sound waves, are collected by the auricle 105 and channeled into and through the ear canal 106. Disposed across the distal end of the ear canal 106 is a tympanic membrane 104 which vibrates in response to the sound signals (i.e., sound waves) 107. This vibration is coupled to the oval window or fenestra ovalis 110 through three bones of the middle ear 102, collectively referred to as the ossicular chain or ossicles 111 and comprising the malleus 112, the incus 113 and the stapes 114. The ossicles 111 of the middle ear 102 serve to filter and amplify the sound signals 107, causing oval window 110 to vibrate. Such vibration sets up waves of fluid motion within the cochlea 116 which, in turn, activates hair cells (not shown) that line the inside of the cochlea 116. Activation of these hair cells causes appropriate nerve impulses to be transferred through the spiral ganglion cells and the auditory nerve 118 to the brain (not shown), where they are perceived as sound.

As noted above, sensorineural hearing loss may be due to the absence or destruction of the cochlea hair cells, which transduce acoustic signals into nerve impulses. One treatment for such hearing loss is a cochlear implant, such as cochlear implant 100 shown in FIGS. 1A and 1B, which bypasses the cochlear hair cells and delivers stimulation (e.g., electrical stimulation) directly to the cochlea nerve cells.

In the illustrative embodiment of FIGS. 1A and 1B, the cochlear implant 100 is a totally implantable cochlear implant, meaning that all components of the cochlear implant are configured to be implanted under skin/tissue 115 of a recipient. Because all components of cochlear implant 100 are implantable, the cochlear implant operates, for at least a finite period of time, without the need of an external device. An external device can be used to, for example, charge an internal power source (battery) of the cochlear implant 100.

The cochlear implant 100 comprises an implant body or main module 120, a lead region 122, and an elongate intra-cochlear stimulating assembly 124. The implant body 120 comprises a hermetically sealed housing 129 in which radio frequency (RF) interface circuitry 132 (sometimes referred to as a transceiver unit), at least one rechargeable battery 134, a signal processor 136, and a stimulator unit 138 are disposed. The implant body 120 also comprises a an internal/implantable coil 130, generally disposed outside of the housing 129, and at least two implantable sensors/transducers 140(A) and 140(B), which may located within the housing 129 or external to the housing 129. As such, although for ease of illustration the implantable sensors 140(A) and 140(B) are shown within housing 129, it is to be appreciated that the implantable sensors 140(A) and 140(B) may have other implanted positions/locations.

The RF interface circuitry 132 is connected to the implantable coil 130 and, generally, a magnet (not shown) is fixed relative to the implantable coil 130. Implantable coil 130 is typically a wire antenna coil comprised of multiple turns of electrically insulated single-strand or multi-strand platinum or gold wire. The electrical insulation of implantable coil 130 is provided by a flexible molding (e.g., silicone molding), which has been omitted from FIG. 1B. In general, the implantable coil 130 and the RF interface circuitry 132 enable the transfer of power and/or data from an external device to the cochlear implant 100. However, it is to be appreciated that various types of energy transfer, such as infrared (IR), electromagnetic, capacitive and inductive transfer, may be used to transfer power and/or data from an external device to a cochlear implant 100 and, as such, FIG. 1B illustrates only one example arrangement.

Elongate stimulating assembly 124 is configured to be at least partially implanted in cochlea 116 and includes a plurality of longitudinally spaced intra-cochlear electrical stimulating contacts (electrical contacts) 142 that collectively form a contact array 144. Stimulating assembly 124 extends through an opening in the cochlea 116 (e.g., cochleostomy 146, oval window 110, the round window 113, etc.) and has a proximal end connected to stimulator unit 138 via lead region 122 that extends through mastoid bone 119. Lead region 122 couples the stimulating assembly 124 to implant body 120 and, more particularly, to stimulator unit 138.

As noted above, the cochlear implant 100 comprises at least two implantable sensors 140(A) and 140(B), where one sensor is more sensitive to body noises than it is to external acoustic sound signals. In the illustrative embodiment of FIG. 1B, the implantable sensor 140(A) is a “sound” sensor/transducer that is primarily configured to detect/receive external acoustic sounds (e.g., an implantable microphone), while the implantable sensor 140(B) is a “vibration” sensor that is primarily configured to detect/receive internal body noises (e.g., another implantable microphone or an accelerometer). For ease of description, embodiments presented herein will be primarily described with reference to the use of an implantable microphone 140(A) as the sound sensor and an accelerometer 140(B) as the vibration sensor. However, it is to be appreciated that these specific implementations are non-limiting and that embodiments of the present invention may be used with different types of implantable sensors.

As noted, the implantable microphone 140(A) and the accelerometer 140(B) may each be disposed in, or electrically connected to, the implant body 120. In operation, the microphone 140(A) and the accelerometer 140(B) detect input (sound/vibration) signals (e.g., external acoustic sounds and/or body noises) and convert the detected input signals into electrical signals. These electrical signals are received by the signal processor 136, which is configured to execute signal processing and coding to convert the electrical signals into processed signals that represent the detected signals. The processed signals are then provided to the stimulator unit 138, which is configured to utilize the processed signals to generate electrical stimulation signals that are delivered to the recipient's cochlea via one or more electrodes 142 implanted in the recipient's cochlea 116. In this way, cochlear implant 100 stimulates the recipient's auditory nerve cells, bypassing absent or defective hair cells that normally transduce acoustic vibrations into neural activity.

It is generally desirable for implantable cochlear implants, such as cochlear implant 100, to be capable of achieving sufficient signal-to-noise ratio (SNR), while being sufficiently insensitive to biological/body noises. As used herein, body noises (BNs) are undesirable sounds induced by the body that are propagated primarily as vibration, such as breathing, scratching, rubbing, noises associated with the movement of the head, chewing, etc. Own voice (OV) (i.e., when the recipient speaks) is a particular case of body noise since the sound is transmitted both through air conduction and bone conduction (i.e., skull vibrations). In certain own voice instances, most of these sound propagates through the skull bones and produce accelerations at the implantable microphone 140(A). That is, in general, the implantable microphone 140(A) is affected by body noises that can be characterized as an acceleration coming from the body (or the recipient's own voice), and captured by the microphone. In the case of own voice, the bone conducted vibrations are loud and, in conventional arrangements, cannot be easily differentiated from other body noises, thus degrading the perception of the recipient's own voice when conventional body noise reduction is performed. As such, there is a need to attenuate own voice and other body noise levels without causing distortion, as perception of own voice is important to speaking well, and long term to exposure to poor own voice perception can negatively affect the way recipients speak. The cochlear implant 100 uses the implantable accelerometer 140(B) to control the vibrations in order to deliver a useful signal to the recipient. That is, in general, the output of the implantable accelerometer 140(B) is used to cancel/attenuate body noises appearing in the output of the implantable microphone 140(A).

In accordance with embodiments presented herein, the output of the implantable accelerometer 140(B) is used for a secondary purpose, namely to obtain a heartbeat signal. The heartbeat signal can be used to measure, for example, a heart rate and intensity of the recipient. in particular, the cochlear implant 100, or a device associated with the cochlear implant (e.g., a computing device, such as mobile phone, tablet computer, fitting system, etc.) is configured to analyze the output of the implantable accelerometer 140(B) (accelerometer signal) to determine/measure heartbeat signal (e.g., measure the heart rate and intensity) which, in turn, can be used to evaluate/monitor cardiac function of the recipient.

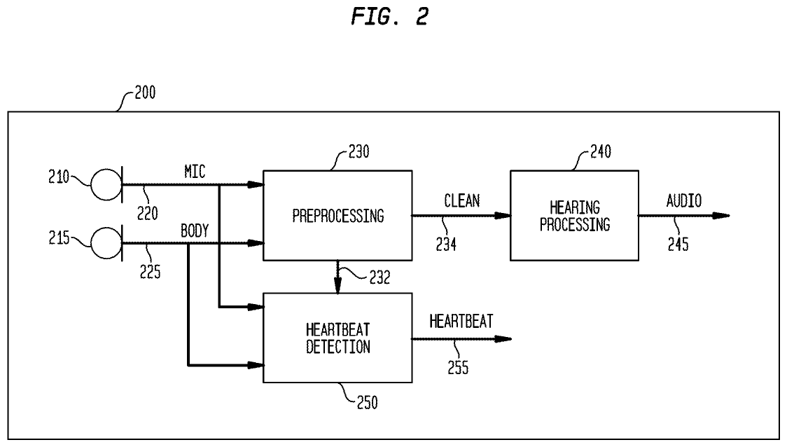

FIG. 2 is block diagram of an exemplary implantable medical device 200 that is configured to generate a heartbeat signal according to the techniques described herein. The device 200 includes a microphone 210 and a vibration sensor 215 that collective obtain input signals for the device 200. The microphone 210 is configured to record microphone signal 220 that is focused on/sensitive to acoustic sound signals (sounds) produced outside the body in which the device 200 is implanted. The vibration sensor 215 is configured to record a body signal 225 that is focused on/sensitive to body noises (e.g., motion and sounds produced within the body) in which the device 200 is implanted. In one example, the vibration sensor 215 may be an accelerometer that measures acceleration of the device 200 as the device 200 is vibrated by different body mechanisms, such as breathing, walking, heartbeat, etc. In another example, the device 200 may be implanted in the head of a recipient and the vibration sensor may be directed toward the skull. Additionally, the vibration sensor 215 may be less sensitive to audio frequency signals as the microphone 210.

The device 200 also includes a preprocessing module 230 that processes the microphone signal 220 and the body signal 225 and generates a noise signal 232. The preprocessing module 230 also generates a “clean” microphone signal 234 from which the body noises have been removed. In one example, the noise signal 232 may be derived from the body signal 225. A hearing processing module 240 further processes the clean microphone signal 234 to generate a processed audio signal 245, which may be them be used to provide stimulation (e.g., acoustical, mechanical, electrical, etc.) to the recipient.

The device 200 further includes a heartbeat detection module 250 that generates a heartbeat signal 255 from inputs including the microphone signal 220, the body noise signal 225, and the noise signal 232. In one example, the heartbeat detection module 250 may determine a body environment to generate a validation signal and determine whether the heartbeat signal 255 is valid. The heartbeat detection module 250 may also be configured to extract features, such as heart rate or blood pressure, from the heartbeat signal 255.

In one example, the device 200 is an totally implantable cochlear implant with a microphone 210 that is sensitive to sound and vibration, and an accelerometer 215 that is sensitive to vibration. Typically, signals from both sensors are used to remove body noise and generate a clean audio signal that may be stimulated in the cochlea of the user. In another example, the device 200 may be a medical device with one or more components configured to be implanted in the head of a recipient for a purpose other than heartbeat detection, such as audio reproduction or body health monitoring. For instance, the device 200 may include a function module directed to detecting body conditions, such as epilepsy detection, sleep apnea detection, tinnitus detection. Alternatively, the device 200 include a function module be directed toward functions of the recipient's ear, such as a cochlear implant, a middle ear implant, a bone conduction device, or a vestibular stimulator.

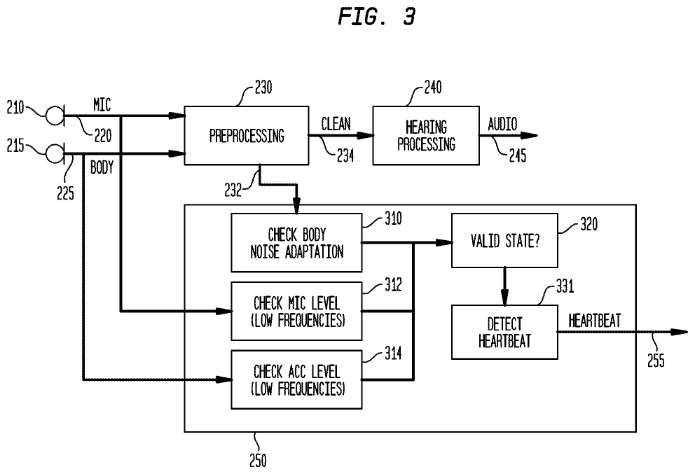

FIG. 3 is a block diagram that further illustrates the operations of the heartbeat detection module 250. The heartbeat detection module 250 includes a module 310 that uses the noise signal 232 from the preprocessing module 230 to evaluate the body environment (e.g., determine what the body noise indicates about the body environment). For instance, the module 310 may determine that the noise signal 232 represents a body environment that is not conducive to recording a valid heartbeat signal, such as when the user is talking. Alternatively, the module 310 may determine that noise signal 232 represents a body environment that may be conducive to recording a valid heartbeat signal, such as simply breathing or external background sounds.

In certain embodiments, the heartbeat detection module 250 can also or alternatively include a module 312 that uses the microphone signal 220 from the microphone 210 to evaluate the body environment. For example, the module 312 can determine whether the microphone signal 220 represents a body environment for a valid heartbeat signal. The module 312 may evaluate, for example, the level (e.g., amplitude) of the microphone signal 220, particularly at low frequencies, to determine whether the body environment is conducive to recording a valid heartbeat signal.

In certain embodiments, the heartbeat detection module 250 can also or alternatively include a module 314 that checks the body signal 225 from the vibration sensor 215 to determine whether the body signal 225 indicates a suitable body environment for a valid heartbeat signal. For instance, the module 314 may detect a high amplitude, low frequency body signal, such as when the user is riding along a bumpy road with strong vibrations, and determine that the heartbeat detection module 250 will not be able to record a valid heartbeat signal. In another example, the body signal 225 may indicate that the walking and determine that the a valid heartbeat signal may be recorded in between the impulse signal generated at each footstep. In other words, the amplitude of the body signal 225 may include a periodically high amplitude indicative of footsteps, which would interfere with recording a valid heartbeat signal, but for the relatively longer time period between footsteps, the heartbeat detection module 250 may be able to record a valid heartbeat signal.

In the example of FIG. 3, a comparison module 320 of the heartbeat detection module 250 collects input from the modules 310, 312, and 314 and makes a final determination on whether the overall body environment allows the heartbeat detection module 250 to record a valid heartbeat signal. In one example, the comparison module 320 may perform additional determinations of the body environment, which may be based on multiple input signals (e.g., the microphone signal 220 and the body signal 225). For instance, the comparison module 320 may determine that a high amplitude microphone signal 220 and a low amplitude body signal is indicative of swimming, and allows the valid detection of a heartbeat signal. As noted, one or more of the modules 310, 312, and 314 could be omitted in certain embodiments. In embodiments in which only one of modules 310, 312, and 314 are present, the comparison module 320 could also be omitted.

Returning to the example of FIG. 3, if the comparison module 320 indicates that the body environment allows for a valid heartbeat signal, a module 330 in heartbeat detection module 250 processes the input signals to generate the valid heartbeat signal 255. In one example, the module 330 filters the body signal 225 to remove audio interference, other body noises, external sounds, or other vibration signals that are not attributed to the heartbeat of the user. The module 330 may also perform a low pass filter or a bandpass filter on the body signal 225 to filter out signals that vary with a frequency too fast or too slow for typical heartbeat signals. For instance, the module 330 may access user settings (e.g., age, gender, and indication of physical fitness) to determine a typical resting heart rate for the user. Alternatively, the module 330 may access previously stored heart rate data for the user to determine an actual resting heart rate based on accumulated data. The cutoff frequency of the low pass or bandpass filter may be based on the resting heart rate of the user.

It is to be appreciated that a number of different filtering methods may be applied to the body signal 225 in order to extract the heartbeat signal 255. For instance, low pass filtering, bandpass filtering, Fast Fourier Transform (FFT) analysis, Kalman filtering, and/or adaptive filtering. Extracting the heart rate and intensity may also use techniques such as a smoothing average, counters, mean, and/or variance.

In another example, the heartbeat detection module 250 may classify the body environment into one or more activity classes, which are correlated with the ability of detecting a valid heartbeat signal. For instance, the body environment may be classified as walking, jogging, sprinting, swimming, cycling, or driving. Different activity classes may be acceptable for generating valid heartbeat signals (e.g., swimming, cycling), unacceptable for generating valid heartbeat signals (e.g., sprinting), or periodically acceptable for generating valid heartbeat signals (e.g., walking).

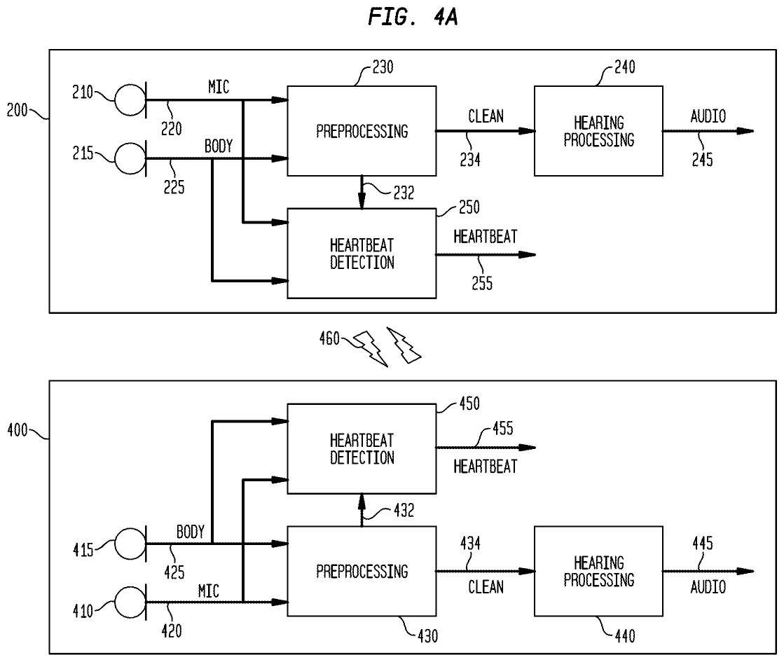

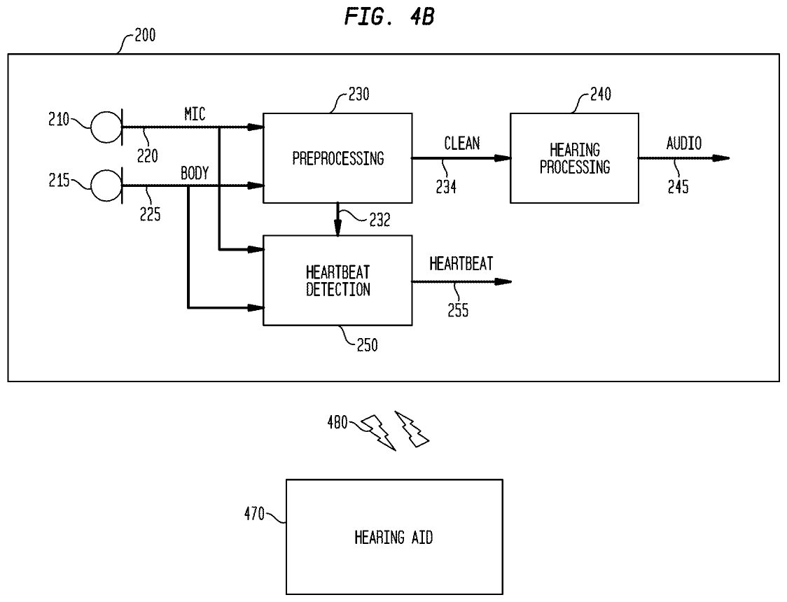

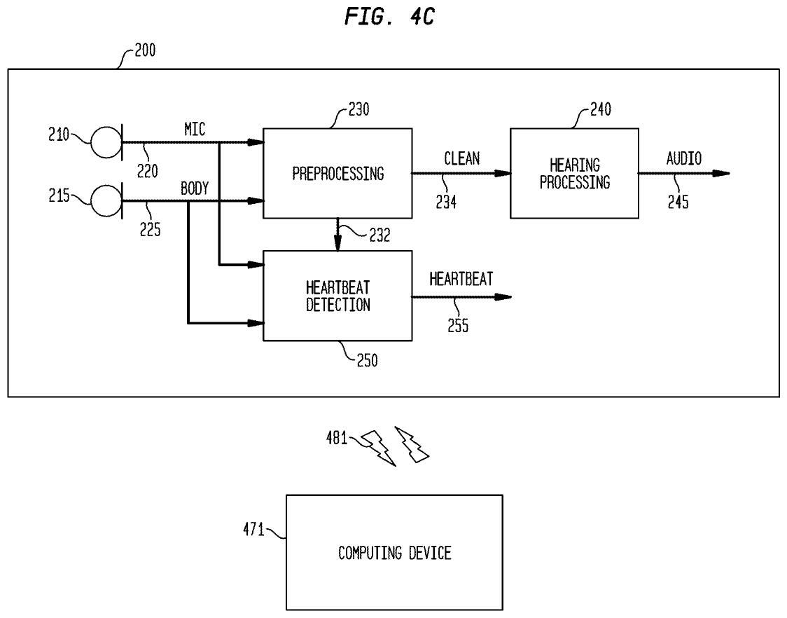

FIG. 4A and FIG. 4B are bock diagrams that illustrate example embodiments in which the implantable medical device 200 communicates with complementary devices to further refine (or partially implement) the detection and generation of a heartbeat signal. In FIG. 4A, the implantable medical device 200 (e.g., a cochlear implant device) is mirrored by a second implantable medical device 400. The second device 400 includes a microphone 410 and vibration sensor 415. Similar to the microphone 210 and the vibration sensor 215 of the first device 200, the microphone 410 generates a microphone signal 420 and the vibration sensor generates a body signal 425.

The second device 400 includes a preprocessing module 430 that removes a noise signal 432 form the microphone signal 220 and generates a clean microphone signal 434. A hearing processing module 440 further processes the clean microphone signal 434 to generate a processed audio signal 445. The second device 400 includes a heartbeat detection module 450 similar to the heartbeat detection module 250 in the first device 200. The heartbeat detection module 450 is configured to generate a valid heartbeat signal 455 from the microphone signal 220, the body signal 225, and the noise signal 232.

The first device 200 and the second device 400 exchange data over a link 460 enabling the first device 200 to compare data from the two devices and produce a final heartbeat signal. In one example, the first device 200 and the second device 400 capture and process signals in parallel and each generate heartbeat signals 255 and 455 separately. The separately generated heartbeat signals 255 and 455 may be compared to determine a final heartbeat signal, which may be provided as output to the user and/or stored for future use.

Alternatively, preliminary data signals (e.g., microphone signal 420, body signal 425, noise signal 432, etc.) may be communicated across the data link 460 from the second device 400 to the first device 200 and processed by one or more modules (e.g., heartbeat detection module 250) in the first device 200. Additionally, the preliminary data signals communicated across the data link 460 may be combined with local data signals (e.g., microphone signal 220, body signal 225, noise signal 232, etc.) to generate a valid heartbeat signal 255. In another example, the second device 400 may be configured to gather data from the first device 200 and perform the processing to generate a valid heartbeat signal 455.

FIG. 4B illustrates an example embodiment in which an external hearing aid 470 communicates with the first device 200 over a communications link 480. The hearing aid 470 may provide additional preliminary signals (e.g., audio signals) that may assist the first device 200 detect a valid heartbeat signal 255. For instance, the hearing aid 470 may provide a clearer audio signal of external sounds than the microphone signal 220, and the heartbeat detection module 250 may use the external audio signal received from the hearing aid 470 over the data link 480 to filter out external sound from the body signal 225 more effectively.

In one example, the valid heartbeat signal 255 from the first device 200 may substantially match the heartbeat signal 455 from the second device 400. When the valid heartbeat signal 255 substantially matches the valid heartbeat signal 455, the valid heartbeat signal 255 and the valid heartbeat signal 455 may be combined and provided as output to the user and/or stored for future use. In a further example, the valid heartbeat signal 255 and the valid heartbeat signal 455 may be compared to determine if one provides a better signal according to a predetermined metric. For instance, the valid heartbeat signal 255 and the valid heartbeat signal 455 may be compared to determine if one provides a higher signal-to-noise ratio than the other. Additionally, properties of the valid heartbeat signal 255 and the valid heartbeat signal 455 (e.g., frequency, absolute magnitude, harmonics, etc.) may be compared to determine whether either or both of the measured heartbeat signals represents a valid state for the physical heartbeat. If the variation between the two measured heartbeat signals is above a predetermined threshold, then both heartbeat signals may be invalidated.

In another example, only one of the first device 200 or the second device 400 provides a valid heartbeat signal, which is provided as output to the user. For instance, the first device 200 may determine that the acquired microphone signal 220 and body signal 225 do not allow for a valid heartbeat signal 255, while the second device 400 determines that the microphone signal 420 and the body signal 425 do allow for a valid heartbeat signal 455. In this example, the single valid heartbeat signal may be provided as output to the user and/or stored for future use.

FIG. 4C illustrates an example embodiment in which an external computing device 471 communicates with the first device 200 over a communications link 481. In certain embodiments, the computing device 471 can provide an indication of the valid heartbeat signal 255 signal to a user (e.g., generate an audible and/or visual representation of the valid heartbeat signal 255. The external computing device 471 can also perform further analysis of the valid heartbeat signal 255 to monitor/evaluate the cardia function of the recipient. It is also to be appreciated that, in certain embodiments, the computing device 471 can perform one or more of the operations described above with reference to the heartbeat detection 250 (e.g., operations of modules 310, 312, 314, 320, and/or 330).

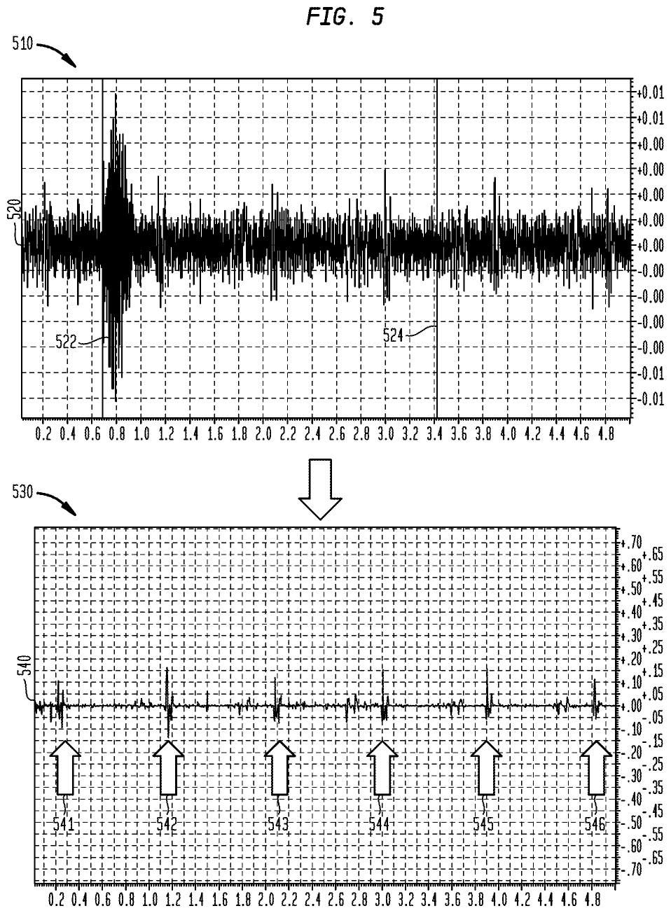

FIG. 5 provides a graphical illustration of filtering a vibration signal to generate a heartbeat signal. The graph 510 shows a vibration signal 520 that includes a portion 522 with own voice interference and a portion 524 with external sound interference. Additionally, breathing sounds throughout the vibration signal 520 further masks the heartbeat signal that is contained within the vibration signal 520. In one example, the vibration signal 520 may already be filtered to relevant frequencies, and the own voice portion 522 and external sound portion 524 are still present in the selected frequency range.

The graph 530 shows a heartbeat signal 540 that results from the processing of the vibration signal 520 according to the techniques describes herein. In one example, the processing may include frequency filtering (e.g., low pass filtering), amplification, and modulation by Hilbert transform envelope detection. The heartbeat signal 540 includes portions 541, 542, 543, 544, 545, and 546 that correspond to individual heartbeats. From the heartbeat signal 540, the heart rate and blood pressure of the recipient may be measured.

In another example, a Kalman filter or extended Kalman filter may be used to provide a robust detection of the heart rate. Known heart rate data may be used to build a state space model of expected heart rate behavior. For instance, resting heart rates of people generally correlate with age, gender, and fitness level, which provides general boundaries for the heart rates of any particular individual. These boundaries may be used with designing a Kalman filter for heartbeat detection.

In a further example, a correlation between heart rate and breathing rate may be exploited to improve the detection of the heart rate. For instance, especially during sleep, the breathing rate of a person affects their heart rate, and a detected breathing rate may be used to refine a model for robust heart rate detection.

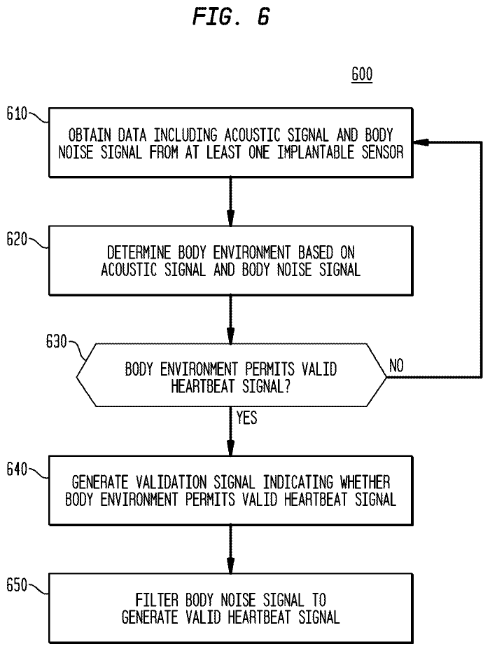

FIG. 6 is a flowchart illustrating operations performed in an example process 600 by an implantable medical device (e.g., device 200) to generate a valid heartbeat signal. At 610, the device obtains data form at least one implantable sensor. The data includes an acoustic signal of external sounds and a body noise signal of internal vibrations. In one example, the device includes a microphone that captures the acoustic signal and an accelerometer that captures the body noise signal.

At 620, the device determines a body environment based on the acoustic signal and the body noise signal. In one example, the body environment is associated with an activity class (e.g., walking, running, swimming, cycling, etc.) that describes the likely activity of the body in which the device is implanted. If the body environment permits the collection of a valid heartbeat signal, as determined at 630, then the device generates a validation signal at 640. In one example, the generation of the validation signal may be correlated with the activity class of the body in which the device is implanted.

At 650, the device filters the body noise signal to isolate a heartbeat signal from any interfering noise signals (e.g., breathing, external sounds, etc.). In one example, the device may only generate the heartbeat signal if there is a validation signal to ensure that the heartbeat signal is valid. Alternatively, the device may attempt to generate the heartbeat signal at 650, but only provide a valid heartbeat signal if the body environment allows the device to generate a validation signal.

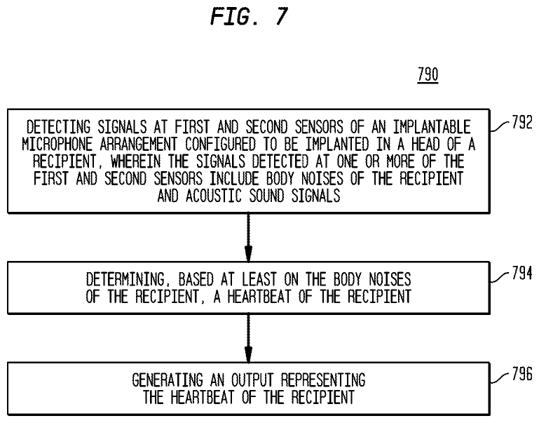

FIG. 7 is a flowchart of an example method 790, in accordance with certain embodiments presented herein. Method 790 begins at 792 where signals are detected at first and second sensors of an implantable microphone arrangement configured to be implanted in a head of a recipient. The signals detected at one or more of the first and second sensors include body noises of the recipient and acoustic sound signals. At 794, a heartbeat of the recipient is determined based at least on the body noises of the recipient. At 796, an output representing the heartbeat of the recipient is generated.

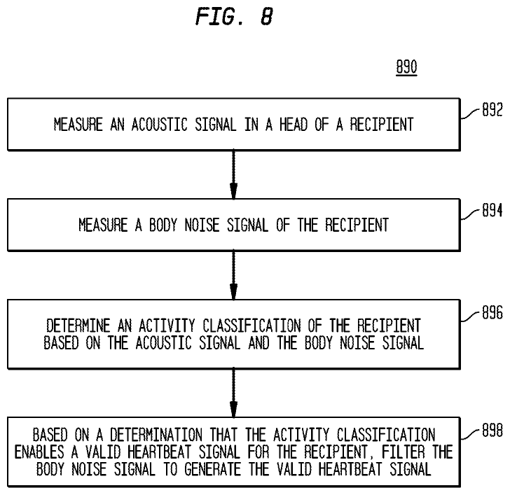

FIG. 8 is a flowchart of an example method 890, in accordance with certain embodiments presented herein. Method 890 begins at 892 where an implantable acoustic sensor configured measures an acoustic signal in a head of a recipient. At 894, an implantable measures a body noise signal of the recipient. At 896, a heartbeat detection module determines an activity classification of the recipient based on the acoustic signal and the body noise signal. At 896, based on a determination that the activity classification enables a valid heartbeat signal for the recipient, the heartbeat detection module filters the body noise signal to generate the valid heartbeat signal. In certain embodiments, the heartbeat detection module can be implanted in the body of the recipient, while in other embodiments the heartbeat detection module can be external to the body of the recipient. In certain embodiments, operations of the heartbeat detection module can be implemented within the body of the recipient, while other aspects of the operations of the heartbeat detection module can be implemented external to the body of the recipient. For example, aspects of the techniques presented herein can be implemented with throat microphones and/or acoustic or vibration sensors located elsewhere in the body (e.g., not in the head of the recipient), as well as with non-implantable sensors, such as mouth microphones, ear canal microphones, etc.

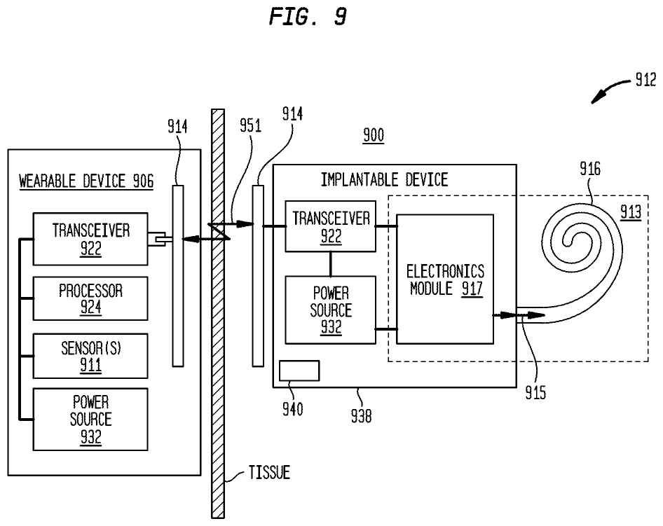

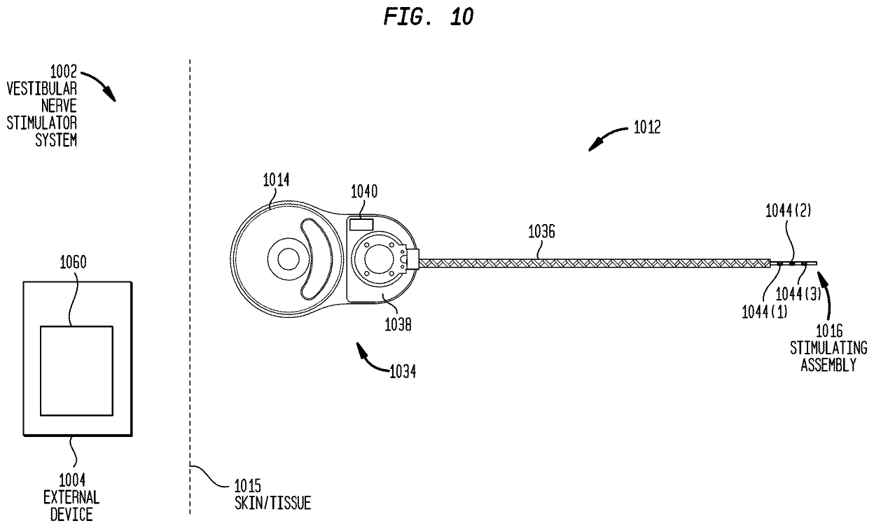

As previously described, the technology disclosed herein can be applied in any of a variety of circumstances and with a variety of different devices. For example, FIGS. 9-11 illustrate example devices configured to determine a heartbeat of a recipient, as described above, in accordance with embodiments presented herein. As described further below, FIG. 9 illustrates an example implantable stimulation system, FIG. 9 illustrates an example vestibular stimulator, and FIG. 10 illustrates a retinal prosthesis. In each of these examples, the respective device includes a vibration sensor implanted in the head of the recipient. The techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, technology described herein can also be applied to consumer devices. These different systems and devices can benefit from the technology described herein.

FIG. 9 is a functional block diagram of an implantable stimulator system 900 that can benefit from the technologies described herein. The implantable stimulator system 900 includes the wearable device 906 acting as an external processor device and an implantable device 912 acting as an implanted stimulator device. In examples, the implantable device 912 is an implantable stimulator device configured to be implanted beneath a recipient's tissue (e.g., skin). In examples, the implantable device 912 includes a biocompatible implantable housing 938. Here, the wearable device 906 is configured to transcutaneously couple with the implantable device 912 via a wireless connection to provide additional functionality to the implantable device 912.

In the illustrated example, the wearable device 906 includes one or more sensors 911, a processor 924, a transceiver 922, and a power source 932. The one or more sensors 911 can be one or more units configured to produce data based on sensed activities. In an example where the stimulation system 900 is an auditory prosthesis system, the one or more sensors 911 include sound input sensors, such as a microphone, an electrical input for an FM hearing system, other components for receiving sound input, or combinations thereof. Where the stimulation system 900 is a visual prosthesis system, the one or more sensors 911 can include one or more cameras or other visual sensors. Where the stimulation system 900 is a cardiac stimulator, the one or more sensors 911 can include cardiac monitors. The processor 924 can be a component (e.g., a central processing unit) configured to control stimulation provided by the implantable device 912. The stimulation can be controlled based on data from the sensor 911, a stimulation schedule, or other data. Where the stimulation system 900 is an auditory prosthesis, the processor 924 can be configured to convert sound signals received from the sensor(s) 911 (e.g., acting as a sound input unit) into signals 951. The transceiver 922 is configured to send the signals 951 in the form of power signals, data signals, combinations thereof (e.g., by interleaving the signals), or other signals. The transceiver 922 can also be configured to receive power or data. Stimulation signals can be generated by the processor 924 and transmitted, using the transceiver 922, to the implantable device 912 for use in providing stimulation.

In the illustrated example, the implantable device 912 includes a transceiver 922, a power source 932, a vibration sensor 940, and a medical instrument 913 that includes an electronics module 917 and a stimulation arrangement 916. The electronics module 917 can include one or more other components to provide medical device functionality. In many examples, the electronics module 917 includes one or more components for receiving a signal and converting the signal into the stimulation signal 915. The electronics module 917 can further include a stimulator unit. The electronics module 917 can generate or control delivery of the stimulation signals 915 to the stimulation arrangement 916. In examples, the electronics module 917 includes one or more processors (e.g., central processing units or microcontrollers) coupled to memory components (e.g., flash memory) storing instructions that when executed cause performance of an operation. In examples, the electronics module 917 generates and monitors parameters associated with generating and delivering the stimulus (e.g., output voltage, output current, or line impedance). In examples, the electronics module 917 generates a telemetry signal (e.g., a data signal) that includes telemetry data. The electronics module 917 can send the telemetry signal to the wearable device 906 or store the telemetry signal in memory for later use or retrieval.

The stimulation arrangement 916 can be a component configured to provide stimulation to target tissue. In the illustrated example, the stimulation arrangement 916 is an electrode assembly that includes an array of electrode contacts disposed on a lead. The lead can be disposed proximate tissue to be stimulated. Where the system 900 is a cochlear implant system, the stimulation arrangement 916 can be inserted into the recipient's cochlea. The stimulation arrangement 916 can be configured to deliver stimulation signals 915 (e.g., electrical stimulation signals) generated by the electronics module 917 to the cochlea to cause the recipient to experience a hearing percept. In other examples, the stimulation arrangement 916 is a vibratory actuator disposed inside or outside of a housing of the implantable device 912 and configured to generate vibrations. The vibratory actuator receives the stimulation signals 915 and, based thereon, generates a mechanical output force in the form of vibrations. The actuator can deliver the vibrations to the skull of the recipient in a manner that produces motion or vibration of the recipient's skull, thereby causing a hearing percept by activating the hair cells in the recipient's cochlea via cochlea fluid motion.

The transceivers 922 can be components configured to transcutaneously receive and/or transmit a signal 951 (e.g., a power signal and/or a data signal). The transceivers 922 can be a collection of one or more components that form part of a transcutaneous energy or data transfer system to transfer the signal 951 between the wearable device 906 and the implantable device 912. Various types of signal transfer, such as electromagnetic, capacitive, and inductive transfer, can be used to usably receive or transmit the signal 951.

Each of the transceivers 922 can include or be electrically connected to a respective coil 914 for the transcutaneous transfer of power and/or data. The power sources 932 can be one or more components configured to provide operational power to other components. The power sources 932 can be or include one or more rechargeable batteries. Power for the batteries can be received from a source and stored in the battery. The power can then be distributed to the other components as needed for operation.

As noted, the implantable device 912 includes a vibration sensor 940. In certain embodiments presented herein, the implantable stimulator system 900 (e.g., implantable device 912 and/or wearable device 906) can operate, as described above, to determine a heartbeat signal from the output of the vibration sensor 940. That is, the implantable stimulator system 900 can include the functionality as described above with reference to FIGS. 2, 3, and/or 4A-4C.

As should be appreciated, while particular components are described in conjunction with FIG. 9, technology disclosed herein can be applied in any of a variety of circumstances. The above discussion is not meant to suggest that the disclosed techniques are only suitable for implementation within systems akin to that illustrated in and described with respect to FIG. 9. In general, additional configurations can be used to practice the methods and systems herein and/or some aspects described can be excluded without departing from the methods and systems disclosed herein.

FIG. 10 illustrates an example vestibular stimulator system 1002, with which embodiments presented herein can be implemented. As shown, the vestibular stimulator system 1002 comprises an implantable component (vestibular stimulator) 1012 and an external device/component 1004 (e.g., external processing device, battery charger, remote control, etc.). The external device 1004 comprises a transceiver unit 1060. As such, the external device 1004 is configured to transfer data (and potentially power) to the vestibular stimulator 1012,

The vestibular stimulator 1012 comprises an implant body (main module) 1034, a lead region 1036, and a stimulating assembly 1016, all configured to be implanted under the skin/tissue (tissue) 1015 of the recipient. The implant body 1034 generally comprises a hermetically-sealed housing 1038 in which RF interface circuitry, one or more rechargeable batteries, one or more processors, and a stimulator unit are disposed. The implant body 1034 also includes an internal/implantable coil 1014 that is generally external to the housing 1038, but which is connected to the transceiver via a hermetic feedthrough (not shown). In this example, the implant body 1034 includes a vibration sensor 1040.

The stimulating assembly 1016 comprises a plurality of electrodes 1044(1)-(3) disposed in a carrier member (e.g., a flexible silicone body). In this specific example, the stimulating assembly 1016 comprises three (3) stimulation electrodes, referred to as stimulation electrodes 1044(1), 1044(2), and 1044(3). The stimulation electrodes 1044(1), 1044(2), and 1044(3) function as an electrical interface for delivery of electrical stimulation signals to the recipient's vestibular system.

The stimulating assembly 1016 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein may be used with stimulating assemblies having different numbers of stimulation electrodes, stimulating assemblies having different lengths, etc.

As noted, the vestibular stimulator 1012 includes a vibration sensor 1040. In certain embodiments presented herein, the vestibular stimulator system 1002 (e.g., vestibular stimulator 1012 and/or external device 1004) can operate, as described above, to determine a heartbeat signal from the output of the vibration sensor 1040. That is, the vestibular stimulator system 1002 can include the functionality as described above with reference to FIGS. 2, 3, and/or 4A-4C.

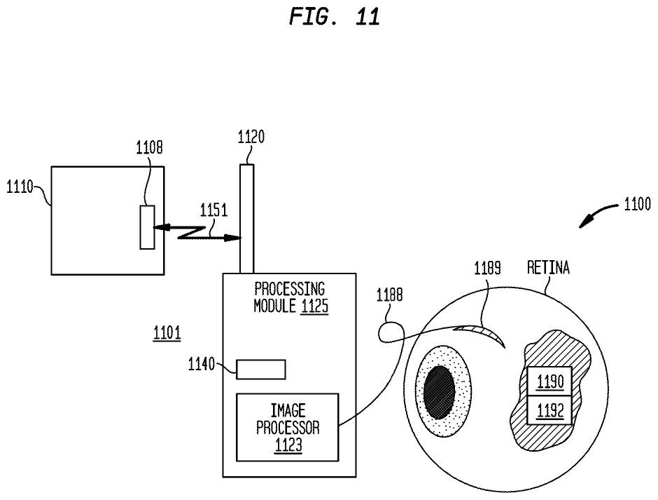

FIG. 11 illustrates a retinal prosthesis system 1101 that comprises an external device 1110 configured to communicate with a retinal prosthesis 1100 via signals 1151. The retinal prosthesis 1100 comprises an implanted processing module 1125 and a retinal prosthesis sensor-stimulator 1190 is positioned proximate the retina of a recipient. The external device 1110 and the processing module 1125 can communicate via coils 1108, 1120. In this example, the processing module 1125 includes a vibration sensor 1140.

In an example, sensory inputs (e.g., photons entering the eye) are absorbed by a microelectronic array of the sensor-stimulator 1190 that is hybridized to a glass piece 1192 including, for example, an embedded array of microwires. The glass can have a curved surface that conforms to the inner radius of the retina. The sensor-stimulator 1190 can include a microelectronic imaging device that can be made of thin silicon containing integrated circuitry that convert the incident photons to an electronic charge.

The processing module 1125 includes an image processor 1123 that is in signal communication with the sensor-stimulator 1190 via, for example, a lead 1188 which extends through surgical incision 1189 formed in the eye wall. In other examples, processing module 1125 is in wireless communication with the sensor-stimulator 1190. The image processor 1123 processes the input into the sensor-stimulator 1190, and provides control signals back to the sensor-stimulator 1190 so the device can provide an output to the optic nerve. That said, in an alternate example, the processing is executed by a component proximate to, or integrated with, the sensor-stimulator 1190. The electric charge resulting from the conversion of the incident photons is converted to a proportional amount of electronic current which is input to a nearby retinal cell layer. The cells fire and a signal is sent to the optic nerve, thus inducing a sight perception.

The processing module 1125 can be implanted in the recipient and function by communicating with the external device 1110, such as a behind-the-ear unit, a pair of eyeglasses, etc. The external device 1110 can include an external light/image capture device (e.g., located in/on a behind-the-ear device or a pair of glasses, etc.), while, as noted above, in some examples, the sensor-stimulator 1190 captures light/images, which sensor-stimulator is implanted in the recipient.

As noted, the processing module 1125 includes a vibration sensor 1040. In certain embodiments presented herein, the retinal prosthesis system 1101 (e.g., retinal prosthesis 1100 and/or external device 1110) can operate, as described above, to determine a heartbeat signal from the output of the vibration sensor 1140. That is, the retinal prosthesis system 1101 can include the functionality as described above with reference to FIGS. 2, 3, and/or 4A-4C.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that.

1. An implantable medical device, comprising:
an implantable microphone arrangement configured to be implanted in a recipient, wherein the implantable microphone arrangement comprises an acoustic sensor configured to detect acoustic signals and a vibration sensor configured to detect vibration signals; and
a heartbeat detection module configured to filter at least the vibration signals to generate a heartbeat signal representing a heartbeat of the recipient.

2. The implantable medical device of claim 1, wherein to filter at least the vibration signals to generate a heartbeat signal, the heartbeat detection module is configured to:
filter the acoustic signals and the vibration signals to generate the heartbeat signal.

3. The implantable medical device of claim 1, wherein the heartbeat detection module is configured to:
determine a body environment based on at least one of the acoustic signals or the vibration signals;
determine whether the body environment permits generation of a valid heartbeat signal; and
generate the heartbeat signal only when the body environment permits generation of a valid heartbeat signal.

4. The implantable medical device of claim 1, further comprising a functional module enabling the implantable medical device to detect body conditions of the recipient or stimulate ear functions of the recipient.

5. The implantable medical device of claim 4, wherein the body conditions include epilepsy, sleep apnea, or tinnitus.

6. The implantable medical device of claim 4, wherein the functional module is configured to stimulate ear functions through a cochlear implant, a middle ear implant, a bone conduction device, or a vestibular stimulator.

7. (canceled)

8. (canceled)

9. (canceled)

10. A method comprising:
obtaining data from at least one sensor, the data comprising an acoustic signal of external sounds and a body noise signal of internal vibrations;
determining a body environment based on the acoustic signal and the body noise signal;
generating a validation signal indicating whether the body environment permits generation of a valid heartbeat signal; and
based on a determination that the validation signal indicates the body environment permits the generation of the valid heartbeat signal, filtering the body noise signal to generate the valid heartbeat signal.

11. The method of claim 10, further comprising providing the valid heartbeat signal to an external device.

12. The method of claim 10, further comprising generating a clean audio signal from the acoustic signal by removing a noise signal derived from the body noise signal.

13. The method of claim 12, wherein determining the body environment comprises determining that the noise signal removed from the acoustic signal exceeds a predetermined threshold.

14. The method of claim 10, wherein determining the body environment comprises comparing an amplitude of at least a portion of the body noise signal to predetermined amplitude threshold.

15. The method of claim 14, wherein the portion of the body noise signal is for frequencies below a frequency cutoff.

16. The method of claim 10, wherein obtaining the data comprises:
obtaining first data from an implantable device, the first data comprising a first acoustic signal and a first body noise signal; and
obtaining second data from a different device, the second data comprising a second acoustic signal and a second body noise signal.

17. The method of claim 16, wherein determining the body environment is based on processing the first acoustic signal and the first body noise signal separately from processing the second acoustic signal and the second body noise signal.

18. The method of claim 17, further comprising:
generating a first validation signal from the processing of the first acoustic signal and the first body noise signal; and
generating a second validation signal from the processing of the second acoustic signal and the second body noise signal.

19. The method of claim 18, wherein filtering the body noise signal to generate the valid heartbeat signal comprises:
selecting a valid body noise signal from either the first body noise signal or the second body noise signal based on a comparison of the first validation signal and the second validation signal; and
filtering the valid body noise signal to generate the valid heartbeat signal.

20. The implantable medical device of claim 1, wherein the heartbeat detection module is configured to:
determine an activity classification of the recipient based on the acoustic signals and the vibration signals; and
based on a determination that the activity classification enables a valid heartbeat signal for the recipient, filter at least the vibration signals to generate the valid heartbeat signal.

21. The implantable medical device of claim 20, further comprising a communications module configured to provide the valid heartbeat signal to an external device.

22. The implantable medical device of claim 21, wherein the communications module is further configured to exchange additional data with a complementary device.

23. The implantable medical device of claim 22, wherein the heartbeat detection module is further configured to determine the activity classification of the recipient based on the additional data from the complementary device, the acoustic signals, and the vibration signals.

24. The implantable medical device of claim 20, wherein the heartbeat detection module is further configured to extract features of the vibration signals to generate the valid heartbeat signal.

25. The implantable medical device of claim 20, wherein the acoustic sensor is a microphone and the vibration sensor is an accelerometer.

26. One or more non-transitory computer readable storage media encoded with software comprising computer executable instructions and when the software is executed operable to cause a processor of an implantable medical device to perform operations including:
obtaining data from at least one sensor of the implantable medical device, the data comprising an acoustic signal of external sounds and a body noise signal of internal vibrations;
determining a body environment based on the acoustic signal and the body noise signal; and
based on a determination that the body environment permits generation of a valid heartbeat signal, filtering the body noise signal to generate the valid heartbeat signal.

27. The one or more non-transitory computer readable storage media of claim 26, wherein the instructions for determining the body environment include instructions for classifying the body environment as either acceptable for generating the valid heartbeat signal or unacceptable for generating the valid heartbeat signal based on an activity class determined from the acoustic signal and the body noise signal.

28. The one or more non-transitory computer readable storage media of claim 26, further comprising instructions for generating a clean audio signal from the acoustic signal by removing a noise signal derived from the body noise signal, wherein the instructions for determining the body environment include instructions for determining that the noise signal removed from the acoustic signal exceeds a predetermined threshold.

29. (canceled)
